(19) United States
(12) Patent Application Publication
(10) Pub. No.: US 2016/0143929 A1
(43) Pub. Date: May 26, 2016

(54) ADMINISTRATION OF GLUFOSFAMIDE FOR THE TREATMENT OF CANCER

(71) Applicant: Threshold Pharmaceuticals, Inc., South San Francisco, CA (US)

(72) Inventor: John G. Curd, Burlingame, CA (US)

(21) Appl. No.: 15/011,113

(22) Filed: Jan. 29, 2016

Related U.S. Application Data
(63) Continuation of application No. 13/058,245, filed on May 4, 2011, now abandoned.
(60) Provisional application No. 61/088,703, filed on Aug. 13, 2008.

(51) Int. Cl. A61K 31/7028; A61K 45/06

(52) U.S. Cl. CPC A61K 31/7028; A61K 45/06

(57) ABSTRACT

Administration of Glufosfamide alone and in combination with another anticancer agent is useful for the treatment of gall bladder cancer.

### REFERENCE TO RELATED APPLICATIONS

This application is continuation of U.S. application Ser. No. 13/058,245, filed May 4, 2011 (pending); which is the U.S. National Stage of PCT/US2009/052370, filed Jul. 31, 2009, published as WO2010/019396 on Feb. 18, 2010; which claims priority to U.S. Provisional Application No. 61/088,703, filed Aug. 13, 2008. The aforelisted PCT and U.S. patents and patent applications are hereby incorporated herein by reference in their entirety for all purposes.

### BACKGROUND OF THE INVENTION

1. Field of the Invention

The present invention relates generally to methods for the treatment of cancer, particularly gall bladder cancer. In particular, the invention relates to methods of treating cancer comprising administration of Glufosfamide alone or in combination with another anticancer agent, and generally relates to the fields of medicine, pharmacology, molecular biology, biology, and chemistry.

2. Description of Related Art

Gall bladder cancer is a serious, life-threatening disease. Like most cancers, the earlier gall bladder cancer is detected, the better the prognosis. However, prognosis is generally very poor: fewer than 10% of diagnosed patients survive more than five years after the initial diagnosis. Treatments generally include surgery and radiation therapy; however, these treatments are generally recommended only for localized gall bladder cancer, which typically is associated only with early stage disease. For later stage disease, chemotherapy is recommended; however, there are no chemotherapeutic treatment regimens widely recognized as effective.

Glufosfamide, also known as beta-D-glucosyl-ifosfamide or beta-D-glucosyl-isophosphoramide mustard or glc-IPM, is a prodrug of an alkylating agent in which isophosphoramide mustard, the cytotoxic metabolite of ifosfamide, is covalently linked to beta-D-glucose. In contrast to ifosfamide, metabolism of Glufosfamide does not release the toxic metabolite acrolein and produces less of the toxic metabolite chloroacetaldehyde. See U.S. Pat. No. 5,622,936, incorporated herein by reference. Glufosfamide has been investigated in several early stage clinical trials as an anticancer agent in the treatment of cancer (see PCT Publication Nos. WO 05/076888, WO 06/071955, WO 06/122227, and WO 07/035961, each of which is incorporated herein by reference). However, only one Phase III clinical trial has been conducted with Glufosfamide. This trial investigated whether Glufosfamide was effective in the treatment of patients with pancreatic cancer who had relapsed after, or not responded to, gemcitabine therapy. The trial did not meet its endpoints, although the data indicated that Glufosfamide did exhibit anticancer activity in some patients (see PCT publication No. WO 08/124691, incorporated herein by reference).

There accordingly remains a need for chemotherapies useful in the treatment of gall bladder cancer, particularly for patients for whom surgery and/or radiation therapy is not indicated. The present invention meets this need by providing novel chemotherapies as summarized below and described in detail herein.

### BRIEF SUMMARY OF THE INVENTION

In one aspect, the present invention provides a method of treating gall bladder cancer comprising administering a therapeutically effective amount of Glufosfamide to a subject in need of such treatment.

In one embodiment, the therapeutically effective amount of Glufosfamide is administered for one or more dosage cycles, each cycle comprising an infusion of Glufosfamide in the range of:

a) about 1.5 to about 8.0 g/m²; about 1.5 to about 6.0 g/m²; about 1.5 to about 4.5 g/m²; about 4.5 to about 8.0 g/m²; about 4.5 to about 6.0 g/m²; or about 4.5 to about 5.0 g/m² over an infusion period of 1-6 hours once every three weeks;

b) about 1.5 to about 3.0 g/m² or about 1.5 to about 2.0 g/m² over an infusion period of 1-6 hours for three consecutive days (days 1, 2 and 3) every three weeks;

c) about 1.5 to about 2.0 g/m² over an infusion period of 1-6 hours once per week; or

d) about 1.5 to about 8.0 g/m²; about 1.5 to about 6.0 g/m²; or about 1.5 to about 4.5 g/m² over an infusion period of 1-6 hours once every four weeks. In one embodiment, the therapeutically effective amount of Glufosfamide is administered for one or more dosage cycles, each cycle comprising a biphasic infusion of Glufosfamide in the range of about 4.5 to about 6.0 g/m² over an infusion period of 6 hours once every three weeks. In one embodiment, the biphasic infusion involves infusing a dose of either 4.5 or 6.0 g/m² in 1000 mL of normal saline, with one quarter of the dose administered during the first 30 min. at a rate of 500 mL/h, and the remainder of the dose being administered over the subsequent 330 min. at a rate of 136 mL/h (with this administration occurring on Day 1 of each three week treatment cycle). In some embodiments Glufosfamide is administered for at least two, at least three or at least four dosage cycles.

In one embodiment, the gall bladder cancer treated is a metastatic, relapsed, or advanced gall bladder cancer.

In another embodiment, the present invention provides a method of treating gall bladder cancer comprising administering Glufosfamide and another anticancer agent to a subject in need of such treatment. In various embodiments, the additional anticancer agent is one or more anticancer agents selected from the group consisting of oxaliplatin, gemcitabine, bortezomib, docetaxel, sorafenib, erlotinib, and lapatinib.

These and other aspects and embodiments are described in detail in the following section.

### DETAILED DESCRIPTION OF THE INVENTION

### Definitions

The following definitions are provided to assist the reader. Unless otherwise defined, all terms of art, notations, and other scientific or medical terms or terminology used herein have the meanings commonly understood by those of skill in the chemical and medical arts. In some cases, terms with commonly understood meanings are defined herein simply for clarity and/or for ready reference, and the inclusion of such definitions herein should not be construed as representing a substantial difference over the definition of the term as generally understood in the art.

“Administering” or “administration of” a drug to a patient (and grammatical equivalents of this phrase) refers to direct administration, which may be administration to a patient by a medical professional or may be self-administration, and/or indirect administration, such as the act of prescribing a drug.

A “subject” or “patient” refers to a mammal in need of treatment for cancer. Generally, the patient is a human. In other embodiments of the invention, however, the patient is a non-human mammal, such as a non-human primate, a dog, cat, cow, horse, rabbit, pig, or the like. In other embodiments of the invention, the patient is an animal such as a mouse or rat, such as an animal commonly used in screening, characterizing, and evaluating drugs and therapies, or a pet, such as a dog or cat.

“Treatment” or “therapy” refers to a method for obtaining beneficial or desired results, including clinical results. For purposes of this invention, beneficial or desired clinical results include, but are not limited to, alleviation or amelioration of one or more symptoms, diminishment of extent of disease, stabilized (i.e., not worsening) state of disease, preventing spread of disease, delaying or slowing of disease progression, amelioration or palliation of the disease state, and remission (whether partial or total), whether detectable or undetectable. “Treatment” can also mean prolonging survival as compared to expected survival in the absence of receiving treatment.

### Treatment Methods

In one aspect, the present invention provides a method of treating gall bladder cancer comprising administering a therapeutically effective amount of Glufosfamide to a subject in need of such treatment. In one embodiment, the therapeutically effective amount of Glufosfamide is administered for one or more dosage cycles, each cycle comprising an infusion of Glufosfamide in the range of:

a) about 1.5 to about 8.0 g/m²; about 1.5 to about 6.0 g/m²; about 1.5 to about 4.5 g/m²; about 4.5 to about 8.0 g/m²; about 4.5 to about 6.0 g/m²; or about 4.5 to about 5.0 g/m² over an infusion period of 1-6 hours once every three weeks;

b) about 1.5 to about 3.0 g/m² or about 0.5 to about 2.0 g/m² over an infusion period of 1-6 hours for three consecutive days (days 1, 2 and 3) every three weeks;

c) about 1.5 to about 2.0 g/m² over an infusion period of 1-6 hours once per week; or

d) about 1.5 to about 8.0 g/m²; about 1.5 to about 6.0 g/m²; or about 1.5 to about 4.5 g/m² over an infusion period of 1-6 hours once every four weeks. Within this embodiment, the therapeutically effective amount of Glufosfamide is administered for one or more dosage cycles, each cycle comprising an infusion of Glufosfamide in the range of about 4.5 to about 6.0 g/m² over an infusion period of 6 hours once every three weeks.

In one embodiment, the therapeutically effective amount of Glufosfamide is administered for one or more dosage cycles, each cycle comprising infusion of Glufosfamide in the range of about 4.5 to about 6.0 g/m² over an infusion period of once every three weeks. In one embodiment, the amount infused is 4.5 g/m². In another embodiment, the amount infused is 6.0 g/m². In one embodiment, the infusion period is 6 hours. In one embodiment, the infusion is a biphasic (fast/slow) infusion. In one embodiment, the biphasic infusion involves infusing a dose of either 4.5 or 6.0 g/m² in 1000 mL of normal saline, with one quarter of the dose administered during the first 30 min. at a rate of 500 mL/h, and the remainder of the dose being administered over the subsequent 330 min. at a rate of 136 mL/h (with this administration occurring on Day 1 of each three week treatment cycle).

In some embodiments Glufosfamide is administered for at least two, at least three or at least four dosage cycles.

In one embodiment, the gall bladder cancer treated is a metastatic, relapsed, or advanced gall bladder cancer. In one embodiment, the gall bladder cancer treated is a gall bladder cancer in a patient for whom surgery and/or radiation therapy is not indicated.

In another embodiment, the present further comprises administering a therapeutically effective amount of another anticancer agent (in addition to Glufosfamide) to the subject in need of such treatment. In one embodiment, the anticancer agent administered in combination with Glufosfamide is selected from the group consisting of bortezomib, capecitabine, carboplatin, cisplatin, docetaxel, doxorubicin, erlotinib, fluorouracil, fluorouridine, gemcitabine, lapatinib, mitomycin, oxaliplatin, and sorafenib, and one or more of such anticancer agents are combined with Glufosfamide for treating a patient with gall bladder cancer. In certain other embodiment, the methods of the present invention are also useful for the treatment of biliary tract cancer, including, without limitation, bile duct cancer. Glufosfamide may be obtained by processes known in the art; see, for example the US and PCT patent publications referenced above in the section entitled “Description of Related Art”, and see PCT publication No. WO 07/146652, incorporated herein by reference).

The present invention arose in part from a Phase I study conducted in Japan to evaluate the safety and pharmacokinetic profile of Glufosfamide administered by intravenous infusion over 6 hours in Japanese patients with solid tumors for whom no effective standard therapy was available. Eligible patients were individuals aged 20 to 75 years who had solid tumors that were either refractory to conventional treatment or for which no standard treatment was available; had an Eastern Cooperative Oncology Group (ECOG) performance status of 0 or 1; had adequate hematepoietic reserves (absolute neutrophil count (ANC) of greater than or equal to 1500/uL, platelet count of greater than or equal to 100,0001uL, hemoglobin concentration of greater than or equal to 9.0 g/dL); had serum total bilirubin and creatinine concentrations of less than or equal to 1.5 times the upper limit of institutional normal (ULN); had serum aspartate aminotransferase and alanine aminotransferase activities of less than or equal to 2.5 times ULN; had not received chemotherapy or radiation therapy within the previous 4 weeks; had no exposure to nitrosoureas or mitomycin within the previous 6 weeks; and had given consent to be hospitalized during the first course of treatment. Patients were ineligible if they had symptomatic brain metastasis; other nonmalignant systemic disease; an active, uncontrolled infection; preexisting nephrotoxicity of grade 3 or 4 (NCI Common Toxicity Criteria—NCI-CTC) resulting from previous therapy; or infection with HIV, HBV, or HCV. They were also ineligible if they were pregnant or nursing, or if they required steroid therapy.

A medical history was obtained from each patient, and physical examinations and routine laboratory evaluations were performed before treatment initiation and weekly thereafter, Chest and other relevant x-rays were obtained during screening and after alternate cycles of treatment. Adverse events were monitored and recorded throughout the study and were graded according to NCI-CTC, version 3.0. The tumor response was assessed for measurable target lesions according to Response Evaluation Criteria in Solid Tumors (RECIST).

This open-label, dose-escalation Phase I study was based on intravenous infusion of the drug in three cohorts of Japanese subjects with malignant solid tumors. Glufosfamide was administered intravenously over 6 h in a total volume of 1000 mL of normal saline at doses of 3200, 4500, and 6000 mg/m². One-quarter of the dose was administered during the first 30 min at a rate of 500 mL/h, with the remainder of the dose being administered over the subsequent 330 min at a rate of 136 mL/h. Glufosfamide was administered on day 1 once every 3 weeks. Antiemetic premedication was not mandatory in the protocol. Granulocyte colony-stimulating factor (G-CSF) was administered for febrile neutropenia, sepsis with neutropenia, or recurrent neutropenia of grade 4.

The starting dose of Glufosfamide was 3200, which was increased to 4500 and then to 6000 in subsequent cohorts of at least three patients. The following adverse events during cycle 1 were defined as DLTs (dose-limiting toxicities): neutropenia of grade 4 (ANC of <500/uL) for >7 days; febrile neutropenia (fever of >38 degrees C. with an ANC of <1000/uL); thrombocytopenia (platelet count of <25,000/uL); nausea or vomiting of grade > (or equal to) 3 despite maximal antiemetic therapy; and any other nonhematologic toxicity of grade > (or equal to) 3 considered drug-related. If any patient experienced a DLT during the first cycle, three additional patients were treated at the same dose. Patients who experienced a DLT could continue Glufosfamide therapy at the preceding dose level. Treatment in subsequent courses was reinitiated only after hematologic recovery (ANC of > (or equal to) 1500/uL, platelet count of > (or equal to) 100,000/uL) and resolution of all other toxicities to grade < (or equal to) 1 or baseline intensity. The MTD (maximum tolerated dose) was defined as the dose at which two or more patients experienced a DLT in the first cycle. The RD (recommended dose) was defined as the dose level immediately below the MTD. In accordance with the methods of the invention, both the RD and MTD (and intermediate doses) can be administered to patients, with the RD preferred for Japanese and other patients of Asian origin and the MID preferred for other patients.

Pharmacokinetic sampling was performed for the first and second cycles. There was substantial interpatient variability in the pharmacokinetics of Glufosfamide after intravenous administration of single doses at all dose levels examined, but both Glufosfamide and IPM exhibit linear pharmacokinetics over the dose range studied. The mean Cₘₐₓ values of Glufosfamide ranged from 107 to 192 ng/mL and were achieved at 2.46 to 3.33 h, with the mean t_{1/2} values ranging from 2.30 to 2.53 h. Glufosfamide exhibited low CLₜₒₜ values (3.47 to 4.08 Lh⁻¹m⁻²) as well as Vss values (8.94 to 9.76 L/m²) that were approximately equal to the volume of extracellular fluid.

Thirteen subjects (nine men and four women; median age, 62 years) were enrolled in the study. Most patients were heavily pretreated, with the median number of prior chemotherapy regimens being three. No DLTs occurred in the patient cohorts treated with the two lowest doses administered; however, two of three treated patients experienced DLTs at the 6000 mg/m² Glufosfamide dose level: one with metabolic acidosis and hypophosphatemia of grade 3, and the other with hypophosphatemia and hypokalemia of grade 3. All DLTs were transient and reversible. The MTD was thus identified as 6000 mg/m², and the RD as 4500 mg/m².

A total of 43 cycles of treatment was administered to 13 patients, with a median of three cycles per patient, and a range of 1 to 11 cycles per patient. Clinically significant effects on ANC or platelet count were rare, and only one patient, treated at the dose of 4500 mg/m², experienced neutropenia of grade 4 without infection, which occurred during cycle 2 and was Short-lived. Red blood cell transfusion was required in one patient treated at the dose of 4500 mg/m² during cycle 3 because of the development of grade 4 anemia. Other hematologic toxicities were mostly grade 1 or 2 and were reversible. The predominant nonhematologic toxicities were fatigue, nausea, a high urinary concentration of beta 2 microglobulin, hypophosphatemia, hypokalemia, and metabolic acidosis. Nonhematelogic toxicities were also generally transient and reversible.

Among all thirteen patients evaluable for response, evidence of antitumor activity was observed in nine patients, with one partial response and eight subjects showing stabilization of disease. Stable disease was confirmed in four patients with colorectal cancer, one with gastric cancer, one with thymic cancer, one with thymoma, and one with non-small cell lung cancer. Surprisingly, a 65-year old female patient with advanced gall bladder cancer who had been previously treated with fluorouracil, cisplatin, and gemcitabine achieved a partial response that persisted for greater than five months after two cycles of treatment with Glufosfamide at 4500 mg/m².

Accordingly, the present invention provides methods for treating cancer, particularly gall bladder cancer, medicaments for use in those methods, and the methods for the manufacture of such medicaments. In one embodiment, the present invention relates to the treatment of gall bladder by the administration of Glufosfamide, alone or in combination with other anticancer agents. In another embodiment, the present invention relates to the treatment of cancer by intravenous infusion of Glufosfamide in Japanese and other patients of Asian origin, where the dose of Glufosfamide is 4500 mg/m², which is lower than the dose of 6000 mg/m² that is well tolerated in Caucasian patients, particularly for the treatment of gall bladder cancer, colorectal cancer, gastric cancer, thymic cancer, thymoma, non-small cell lung cancer, and biliary duct cancer.

As noted above Glufosfamide may be administered alone or in combination with another anticancer agent, Exemplary therapeutic agents, and their modes of administration, for treating gall bladder cancer are described for example in the reference Hiroshi at al., Oncology, 2004, 66 (2): 138-142 (incorporated herein by reference). Additionally, the therapeutically effective amount of a drug other than Glufosfamide that is administered in accordance with the present invention is known to physicians and provided, for example and without limitation, in the product descriptions found in the most recent editions of the PHYSICIANS' DESK REFERENCE, Medical Economics Company, Inc., Oradell, N.J.; and Goodman & Gilman's THE PHARMACOLOGICAL BASIS OF THERAPEUTICS”, McGraw-Hill, New York; and/or are available from the Federal Drug Administration and/or are discussed in the medical literature. Therapeutically effective amounts and methods of administering Glufosfamide for the treatment of other cancers are described in the references U.S. Pat. Nos. 5,622,936 and 6,489,302 and PCT Publication Nos. WO 05/076888, WO 06/071955, WO 06/122227, and WO 07/035961 and can be adapted for the present inventions by those of skill in the art upon reading this disclosure.

While the present invention has been described with reference to the specific embodiments thereof, those skilled in the art will understand that various changes can be made and equivalents can be substituted without departing from the scope of the invention. In addition, many modifications can be made to adapt a particular situation, material, composition of matter, process, process step or steps, to achieve the benefits provided by the present invention without departing from the scope of the present invention. All such modifications are intended to be within the scope of the claims appended hereto. All publications and patent documents cited herein are incorporated herein by reference as if each such publication or document was specifically and individually indicated to be incorporated herein by reference. Citation of publications and patent documents is not intended as an indication that any such document is pertinent prior art, nor does it constitute any admission as to the contents or date of the same.

1. A method of improving prognosis of a human patient who has gall bladder cancer, the method comprising administering to the patient a therapeutically effective amount of glufosfamide.

2. The method of claim 1 wherein the therapeutically effective amount of glufosfamide is administered for one or more dosage cycles, each cycle comprising an infusion of glufosfamide in the range of:
a) about 1.5 to about 8.0 g/m²; about 1.5 to about 6.0 g/m²; about 1.5 to about 4.5 g/m²; about 4.5 to about 8.0 g/m²; about 4.5 to about 6.0 g/m²; or about 4.5 to about 5.0 g/m² over an infusion period of 1-6 hours once every three weeks;
b) about 1.5 to about 3.0 g/m² or about 1.5 to about 2.0 g/m² over an infusion period of 1-6 hours for three consecutive days (days 1, 2 and 3) every three weeks;
c) about 1.5 to about 2.0 g/m² over an infusion period of 1-6 hours once per week; or
d) about 1.5 to about 8.0 g/m²; about 1.5 to about 6.0 g/m²; or about 1.5 to about 4.5 g/m² over an infusion period of 1-6 hours once every four weeks.

3. The method of claim 2 wherein the therapeutically effective amount of glufosfamide is administered for one or more dosage cycles, each cycle comprising an infusion of glufosfamide in the range of about 4.5 to about 6.0 g/m² over an infusion period of 6 hours once every three weeks.

4. The method of claim 3 wherein the gall bladder cancer treated is relapsed gall bladder cancer.

5. The method of claim 1 further comprising administering an anticancer agent other than glufosfamide.

6. The method of claim 1, wherein the glufosfamide is administered as a monotherapy.

7. A method of treating gall bladder cancer in a human patient in need thereof, comprising administering to the patient a therapeutically effective amount of glufosfamide, optionally in combination with a chemotherapeutic agent other than gemcitabine.

8. A second line therapy for treating gall bladder cancer in a patient, wherein the cancer has previously failed treatment with one or more other chemotherapeutic agents, the therapy comprising administering to the patient a therapeutically effective amount of glufosfamide.

9. The therapy of claim 8, wherein the gall bladder cancer in the patient was previously unresponsive to therapy with gemcitabine.

10. The therapy of claim 8, wherein the gall bladder cancer has relapsed in the patient after previous therapy with gemcitabine.

11. The therapy of claim 8, wherein the gall bladder cancer in the patient has not responded to three prior chemotherapy regimens.

12. The therapy of claim 8, wherein the gall bladder cancer in the patient is refractory to fluorouracil, cisplatin, and gemcitabine.

13. The therapy of claim 8, wherein the glufosfamide is administered as a monotherapy.

14. A method of treating gall bladder cancer in a patient in need thereof, comprising administering to the patient a therapeutic dose of glufosfamide in one or more dosage cycles, each cycle comprising an infusion of glufosfamide of at least about 6.0 g/m².
